(19) United States
(12) Patent Application Publication
(10) Pub. No.: US 2015/0272558 A1
(43) Pub. Date: Oct. 1, 2015

(54) DEVICES, METHODS AND KITS FOR TISSUE APPROXIMATION

(71) Applicant: Boston Scientific Scimed, Inc., Maple Grove, MN (US)

(72) Inventors: Paul Smith, Smithfield, RI (US); Dylan Murphy, Walpole, MA (US); Robert B. DeVries, Northboro, MA (US); Michelle Fater, Worcester, MA (US)

(73) Assignee: Boston Scientific Scimed, Inc., Maple Grove, MN (US)

(21) Appl. No.: 14/667,863

(22) Filed: Mar. 25, 2015

Related U.S. Application Data
(60) Provisional application No. 61/969,895, filed on Mar. 25, 2014.

(51) Int. Cl. A61B 17/00; A61B 17/06; A61B 17/04

(52) U.S. Cl. CPC A61B 17/0057; A61B 17/00491; A61B 17/0401; A61B 17/06066; A61B 2017/0065; A61B 2017/00663; A61B 2017/0464; A61B 2017/06052

(57) ABSTRACT

The present disclosure pertain to methods of tissue approximation of a tissue opening in a tissue wall of a body lumen. The method comprises pushing a suitable device through the tissue wall near at least one edge of the opening and pulling opposing edges of the tissue opening nearer to one another or into contact with one another. Also described are devices and kits that are useful for approximation of a tissue opening in a tissue wall of a body lumen.

### STATEMENT OF RELATED APPLICATION

This application claims the benefit of U.S. provisional application Ser. No. 61/969,895, filed Mar. 25, 2014, entitled “DEVICES, METHODS AND KITS FOR TISSUE APPROXIMATION”, the disclosure of which is hereby incorporated by reference in its entirety.

### FIELD OF THE DISCLOSURE

The disclosure relates to devices, methods and kits for tissue approximation.

### BACKGROUND OF THE DISCLOSURE

Pathologies of the gastro-intestinal (GI) system, the biliary tree, the vascular system and other body lumens and hollow organs are often treated through endoscopic procedures, many of which require active and/or prophylactic hemostasis. Hemostatic devices are often deployed via endoscopes are often used to stop internal bleeding, for example, by clamping or otherwise joining together edges of a wound or incision. For example, hemostatic clips grasp tissue surrounding a wound holding edges of the wound together until natural healing processes have closed the wound.

In certain cases, however, the dimension of the wound or incision is such that it would be desirable to approximate the tissue (i.e., it would be desirable to bring the edges of the tissue nearer to one another or together) prior to deploying a clip or some other more permanent closure solution.

### SUMMARY OF THE DISCLOSURE

In accordance with various aspects of the disclosure, methods of tissue approximation of a tissue opening in a tissue wall of a body lumen of a patient are provided. The methods comprise pushing a suitable device through the tissue wall near at least one edge of the opening and pulling opposing edges of the tissue opening nearer to one another or into contact with one another. In certain embodiments, the device comprises a suture having first and second ends and a tissue anchor secured to the first end. In certain other embodiments, the device comprises an angled or curved needle.

In various aspects, methods for tissue approximation of a tissue opening in a tissue wall of a body lumen having an inner luminal tissue wall surface and an outer abluminal tissue wall surface are provided. The methods comprise (a) providing a device selected from either (i) a first device comprising a suture having first and second ends and a tissue anchor secured to the first end or (ii) a second device comprising an angled or curved needle, (b) pushing the device through the tissue wall near at least one edge of the opening, and (c) pulling opposing edges of the tissue opening nearer to one another or in contact with one another.

In various embodiments, which may be used in combination with the above aspects and embodiments, the selected device is the first device and the method comprises (a) attaching the anchor to the inner luminal tissue wall surface near a first edge of the tissue opening, (b) pushing the second end through the tissue wall near a second edge of the tissue opening opposite the first edge such that the second end is positioned beyond the outer abluminal tissue wall surface, and (c) pulling the second end through the opening such that it is positioned inside the inner luminal tissue wall surface, wherein the second end is pulled until the first and second edges of the tissue opening are drawn nearer to one another or in contact with one another. For example, the anchor may be an adhesive pad that adheres to the luminal tissue wall surface, or the anchor may be an anchor that pierces the luminal tissue wall surface, among other possibilities.

In various embodiments, which may be used in combination with the above aspects and embodiments, the methods may further comprise applying an agent selected from a hemostatic agent, an adhesive agent and a hemostatic adhesive agent to the tissue.

In various embodiments, the present disclosure pertains to kits for performing the above methods. The kits may comprise: (a) the first device and (b) one or more of the following: (i) a tubular sharp-tipped suture delivery device, (ii) a hemostatic agent, adhesive agent or hemostatic-adhesive agent, (iii) one or more tissue closure devices, and (iv) one or more injectable agents to assist with tissue approximation.

In various embodiments, which may be used in combination with the above aspects and embodiments, the device selected is the second device and the method comprises (a) pushing a tip of the needle through the tissue wall near a first edge of the tissue opening, (b) pulling the first edge toward a second edge of the opening opposite the first side, and (c) pushing the tip of the needle through the tissue wall near the second edge, such that the needle approximates the first and second edges.

In various embodiments, which may be used in combination with the above aspects and embodiments, the second device comprises an elongated member secured to the needle. In certain of these embodiments, the method further comprises severing the needle from the elongated member after the needle approximates the first and second edges.

In various embodiments, which may be used in combination with the above aspects and embodiments, the methods may further comprise applying an agent selected from a hemostatic agent, an adhesive agent and a hemostatic adhesive agent to the tissue.

In various embodiments, which may be used in combination with the above aspects and embodiments, the needle may be hollow and the agent may be applied from an aperture in a side of the needle.

In various embodiments, the present disclosure pertains to kits for performing the above methods. The kits may comprise: (a) the second device and (b) one or more of the following: (i) a hemostatic agent, adhesive agent or hemostatic-adhesive agent, (ii) one or more tissue closure devices and (iii) one or more injectable agents to assist with tissue approximation.

In various aspects, which may be used in combination with the above aspects and embodiments, a device is provided which comprises an elongated member and an angled or curved needle secured to the elongated member.

In certain embodiments, which may be used in combination with the above aspects and embodiments, the needle may be angled or curved by at least 180°.

In various embodiments, which may be used in combination with the above aspects and embodiments, the needle may be detachable from the elongated member. The needle may be, for example, frangible upon application of mechanical or electrical energy.

In various embodiments, which may be used in combination with the above aspects and embodiments, the (a) the elongated member is a tubular member comprising a first lumen and (b) the needle is hollow and comprises (i) a second lumen in communication with the first lumen and (ii) an aperture in a side of the needle, the aperture connecting the second lumen with an exterior of the needle.

In various aspects, which may be used in combination with the above aspects and embodiments, a device is provided which comprises a suture attached to a tissue anchor. May be for example, an adhesive anchor or a tissue-penetrating anchor, among other possibilities.

These and many additional aspects and embodiments of the present disclosure will become immediately apparent to those of ordinary skill in the art upon review of the Detailed Description and any appended claims to follow.

### BRIEF DESCRIPTION OF THE DRAWINGS

FIG. 1 is a partial schematic view of a method of tissue approximation and hemostasis, in accordance with an embodiment of the present disclosure.

FIG. 2 is a partial schematic view of a method of tissue approximation and hemostasis, in accordance with another embodiment of the present disclosure.

FIGS. 3A and 3B are schematic illustrations of a method of tissue approximation and hemostasis, in accordance with another embodiment of the present disclosure.

### DETAILED DESCRIPTION

A more complete understanding of the present disclosure is available by reference to the following detailed description of numerous aspects and embodiments of the disclosure. The detailed description of the disclosure which follows is intended to illustrate but not limit the disclosure.

The present disclosure is directed to methods, devices and kits for approximating the tissue surrounding a tissue opening (e.g., an opening associated with a natural wound, defect, incision, perforation, etc.), and is particularly useful for approximating the tissue surrounding an opening in the GI tract.

In certain embodiments, the methods, devices and kits of the present disclosure are used to shrink the working diameter of a tissue opening, prior to implementing a more permanent closure solution, such as the introduction of a clip, suture, staple, or other closure device.

In certain embodiments, devices are provided which are adapted to deliver a hemostatic agent to tissue. Examples of hemostatic agents include surgical adhesives such as cyanoacrylate glues and agents that activate based on body chemistry. Cyanoacrylate glues include alkyl cyanoacrylates, for example, C1-C10 alkyl cyanoacrylates such as n-butyl-2-cyanoacrylate and 2-octyl-cyanoacrylate. Examples of agents that activate based on body chemistry include thrombin (e.g., bovine thrombin, pooled human plasma derived thrombin, recombinant human thrombin, etc.). Other examples of agents that activate based on body chemistry include gelatin products such as porcine gelatin products (e.g., Surgiflo®) and bovine collagen (e.g., Avitene®). In some embodiments, gelatin products, such as porcine gelatin and bovine gelatin, among others, may be mixed with thrombin prior to administration. Other known agents include oxidized regenerated cellulose and starch (e.g., Arista® Microporous Polysaccharide Hemospheres). Still other agents include those that become activated upon exposure to enzymes present in the GI tract or upon exposure to changes in temperature, for instance, reverse thermosensitive polymers such as poloxamers or poloxamines.

In certain aspects of the present disclosure, a tissue wall is penetrated on one or both sides of a tissue opening and the two sides are approximated using a suitable medical article.

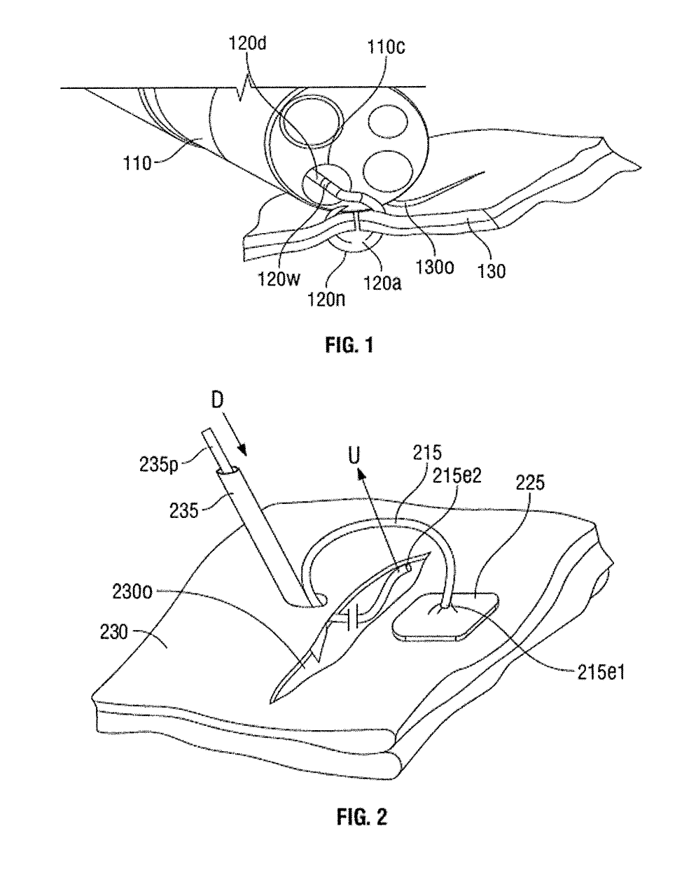

In this regard, FIG. 1 is a schematic illustration of a procedure for tissue approximation and hemostasis, in accordance with an embodiment of the present disclosure. Turning to FIG. 1, an endoscope 110 (partial view) is shown adjacent to an opening 130o in a tissue wall 130. Extending from one working channel 110c of the endoscope 110 is a medical article for tissue approximation, specifically, a device 120d terminating in a non-linear needle 120n. The non-linear needle 120n may be, for instance, a curved needle having a curvature of 180° or more, such as a hook shaped needle or a needle having a full loop, among others. In some embodiments, the needle may have the curved shape only after being released from the delivery device (see below). It is noted that while a curved needle is shown, other non-linear needles such as bent or angled needles (e.g., a needle having multiple bending sites, etc.) may also be employed.

During the procedure, the needle is pushed through the tissue wall 130 near one edge of the tissue opening 130o (the right-hand side when viewing FIG. 1) effectively hooking the edge of the tissue opening 130o, thereby allowing the edge of the tissue opening 130o to be drawn toward the opposing edge of the tissue opening 130o (the left-hand edge when viewing FIG. 1). By penetrating the tissue wall 130 on both sides of the tissue opening 130o, the needle 120n can be used to provide a temporary tack for achieving tissue approximation, thereby allowing a supplemental closure technique to be performed. Examples of supplemental closure techniques include, for example, the application of tissue clips, sutures or other closure devices or the application of a tissue adhesive and/or a hemostatic agent. The needle 120n can then optionally be removed from the tissue at that point.

In some embodiments, the needle 120n can be used to semi-permanently secure the tissue opening 130o in an approximated orientation until the healing process is complete. In such embodiments, the needle 120n may be configured to be frangible, for instance, by providing a region 120w that is predisposed to allow separation the needle 120n from the remainder of the device 120d, for example, by application of mechanical energy, heat energy or another form of energy. For instance, the region 120w may be a thinned (e.g., necked down or notched) region that allows the distal end to be mechanically snapped off the remainder of the needle, for example, by twisting, bending, pulling, and so forth. In other embodiments, the region 120w may be a conductive region that melts upon application of a suitable current, thereby releasing the distal end of the needle. The conductive region may preferentially melt, for example, because it has a lower melting point than other portions of the needle (e.g., due to its chemical composition) or because it is a thinner region leading to higher current density (and thus temperature) than other portions of the device. Regardless of the mechanism, once the needle 120n has been severed, the remainder of the device 120d can be removed, freeing the channel for other uses.

In some embodiments, portions of the device 120d, including the needle 120n are hollow, allowing delivery of a suitable agent from the needle 120n. In certain embodiments, the needle 120n is provided with an aperture 120a, for example, on the side of the needle lying on the inside of the bend or curve of the needle (e.g., at or near a midpoint of the bend or curve, among other possibilities). This allows a suitable agent, such as an adhesive agent, hemostatic agent or adhesive-hemostatic agent, to be applied to the tissue junction. The agent may be in any suitable fluid form, for example, in the form of a flowable powder, slurry, dispersion, suspension, solution, or other fluid.

Kits may be provided for forming the above-described procedure. Such kits may contain, for example, any combination of two or more of the following items: (a) a device having a needle, like that previously described, (b) a hemostatic agent, adhesive agent or hemostatic-adhesive agent (e.g., disposed in a syringe, catheter, vial, ampoule, or other container), (c) one or more sutures, clips, staples, or other tissue closure devices, (d) one or more injectable agents to assist with tissue approximation such as injectable bulking agents or injectable magnetic agents (discussed below), (e) Surgiflo® Hemostatic Matrix or Surgicel® Fibrillar™ Absorbable Hemostat, produced by Ethicon, and (f) printed material including storage information and/or instructions regarding how to use the kit in a medical procedure.

Another embodiment of a procedure in which a tissue wall is penetrated on one or both sides of a tissue opening, and the two sides are approximated using a suitable medical article, is illustrated in FIG. 2. In this regard, FIG. 2 is a schematic illustration of a suture-based procedure for tissue approximation and hemostasis. The procedure illustrated in FIG. 2 may be performed, for example, with the assistance of an endoscope (not shown). In FIG. 2, a first end 215e1 of a suture 215 is attached to tissue 230 adjacent to an edge of an opening 230o in the tissue by means of a suitable tissue anchor 225.

In the embodiment shown, the tissue anchor 225 is an adhesive member which is adhered to the tissue. Other examples of tissue anchors include tissue penetrating anchors such as a hook anchor, a barbed anchor, or a loop anchor.

After attaching the first end 215e1 of the suture 215 via the anchor 225, a sharp-tipped tubular suture delivery device 235 containing a second end 215e2 of the suture 215 is pressed through the tissue 230 near an edge of the tissue opening 230o opposite the edge where the first end 215e1 of the suture 215 is attached. After penetrating the wall of the tissue 230, the second end 215e2 of the suture 215 is expelled from the suture delivery device 235. The second end 215e2 of the suture 215 may be pre-severed or severed from a longer length of suture during the procedure. In FIG. 2, the second end 215e2 of the suture 215 is expelled from the suture delivery device 235 by pushing a push rod 235p in direction D relative to the suture delivery device 235, among other options.

After expulsion of the second end 215e2 of the suture 215 on the opposite (abluminal) side of the wall of the tissue 230, the suture delivery device 235 may be removed. The second end 215e2 of the suture 215 can then be grasped through the tissue opening 230o using a suitable grasping device (e.g., forceps, etc.) and pulled in a direction U, away from the tissue opening 230o, drawing opposing edges of the opening 230o in the tissue wall 230 toward one another, thereby closing or reducing the size of the opening 230o.

By holding the suture 215 in this position, the suture 215 can be used to temporarily achieve tissue approximation while a supplemental closure technique is performed. Examples of supplemental closure techniques include, for example, the application of tissue clips, sutures or other closure devices or the application of an adhesive agent (which may also be a hemostatic agent). The suture 215 and anchor 225 can then optionally be removed from the tissue 230 at that point.

Alternatively, the suture 215 can be used to semi-permanently achieve tissue approximation (e.g., at least until the healing process is complete) by tying off the suture. Where an endoscope is employed, this allows the suture grasping device to be removed, freeing up a working channel of the endoscope. In such embodiments, the suture 215 and/or anchor 225 may optionally be bioerodible. In such embodiments, a supplemental closure technique may be performed, a hemostatic agent may be applied, or both.

In certain embodiments, suture 215 may be permeated with or coated with a suitable adhesive agent, hemostatic agent or adhesive-hemostatic agent.

Kits may be provided for forming the proceeding procedure. Such kits may contain, for example, any combination of two or more of the following: (a) a suture with tissue anchor, (b) a suture delivery device, for example, a sharp-tipped tubular suture delivery device like that previously described, (c) a hemostatic agent, adhesive agent or hemostatic-adhesive agent (e.g., disposed in a syringe, catheter, vial, ampoule, or other container), (d) one or more clips, staples, or other tissue closure devices, (e) one or more injectable agents to assist with tissue approximation such as injectable bulking agents or injectable magnetic agents (described below), (f) Surgiflo® Hemostatic Matrix or Surgicel® Fibrillar™ Absorbable Hemostat: produced by Ethicon, and (g) printed material including storage information and/or instructions regarding how to use the kit in a medical procedure.

Additional embodiments of the present disclosure, which may be used in conjunction with or independently of other procedures described elsewhere herein, will now be described.

Some of these additional embodiments pertain to the use of electrical field stimulation to excite and cause temporary contraction of tissue. See, e.g., R. Axelrod et al., “Response of colonic smooth muscle from newborn and adult rabbits to electrical field stimulation,” Pediatr Res. 1994 Apr; 35(4 Pt 1): 470-3. This process is akin to other electrical stimulation processes such as electrical muscle stimulation (EMS) or transcutaneous electrical nerve stimulation (TENS). In this process, one or more electrodes are attached to tissue adjacent to a tissue opening. In some embodiments, one or more additional electrodes (e.g., counter-electrodes) may also be placed in contact with the patient remote from the tissue opening. Suitable electrodes include conductive pad electrodes and tissue-penetrating electrodes such as corkscrew electrodes, among others. Application of a suitable electrical potential to properly placed electrodes is then used to contract the smooth muscle tissue in the area of the tissue opening, leading to tissue approximation. As previously noted, such a procedure may facilitate the implementation of additional tissue approximation techniques as described herein.

Some of these additional embodiments pertain to the use of injectable agents, one example of which is a temporary or permanent bulking agent. One can inject a bulking agent in order to swell and at least partially close a tissue opening. Examples of bulking agents include injectable glass or ceramic particles (e.g., glass or ceramic beads). Examples of bulking agents further include injectable polymeric agents such as polymeric beads (e.g., polyvinyl alcohol (PVA) or alginate hydrogel beads) or hydrophilic polymers (e.g., polysaccharides such as xanthan gum, sodium carboxymethyl cellulose, hyaluronic acid, etc.) dispersed in water, among others.

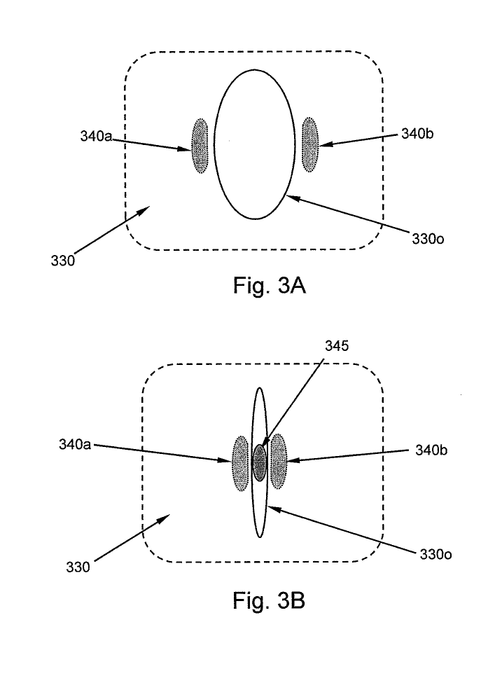

Another example of an injectable agent is an injectable magnetic agent. Examples of magnetic agents include injectable particles of magnetic material including ferromagnetic materials (e.g., iron, nickel, cobalt and certain rare earth elements such as gadolinium, dysprosium, etc.) and ferrimagnetic materials (e.g., magnetite, maghemite and various ferrites including nickel ferrite, cobalt ferrite, manganese ferrite, nickel zinc ferrite, manganese zinc ferrite, etc.). In this regard, FIGS. 3A and 3B are schematic illustrations of a procedure for tissue approximation, in accordance with an embodiment of the present disclosure. The procedure illustrated in FIGS. 3A and 3B may be performed, for example, with an endoscope (not shown).

As an initial step, magnetic particles are injected into tissue 330 adjacent to an opening 330o on opposite sides of the opening 330o, thereby forming magnetic particle implants 340a, 340b. After the magnetic particle implants 340a, 340b are formed in the tissue 330, a magnet 345 may be placed in the tissue opening 330o between the magnetic particle implants 340a, 340b. The magnetic field exerted by the magnet 345 exerts an attractive force on the magnetic particles in the magnetic particle implants 340a, 340b, drawing the magnetic particle implants 340a, 340b toward the magnet (and thus toward one another), thereby approximating the tissue associated with the tissue opening 330o as shown in FIG. 3B.

The magnet 345 may be selected, for example, from a variety of temporary magnets, permanent magnets and electromagnets, with an electromagnet being particularly beneficial in some embodiments, because the magnetic field can be created once the electromagnet is in place. In the embodiment shown, the magnetic particles may or may not be magnetized. If the particles are magnetized, a separate magnet may not be required to draw the implants together.

Each of the tissue approximation procedures described herein may be performed in conjunction with an endoscopic procedure. Endoscopic procedures are commonly accompanied by introducing an insufflation gas into a body lumen (e.g., a gastrointestinal tract) being examined. Various tissue approximation procedures described herein, may be assisted by reducing or ceasing the flow of insufflation gas or by exerting temporary suction to release insufflation tension.

Although various embodiments are specifically illustrated and described herein, it will be appreciated that modifications and variations of the present disclosure are covered by the above teachings and are within the purview of any appended claims without departing from the spirit and intended scope of the disclosure.

1. A method for tissue approximation of a tissue opening in a tissue wall of a body lumen having an inner luminal tissue wall surface and an outer abluminal tissue wall surface, said method comprising (a) providing a device selected from either (i) a first device comprising a suture having first and second ends and a tissue anchor secured to said first end or (ii) a second device comprising an angled or curved needle, (b) pushing said device through the tissue wall near at least one edge of said opening and (c) pulling opposing edges of said tissue opening nearer to one another or in contact with one another.

2. The method of claim 1, wherein the selected device is said first device and wherein said method comprises (a) attaching said anchor to the inner luminal tissue wall surface near a first edge of said tissue opening, (b) pushing said second end through the tissue wall near a second edge of said tissue opening opposite the first edge such that the second end is positioned beyond the outer abluminal tissue wall surface, and (c) pulling said second end through said opening such that it is positioned inside the inner luminal tissue wall surface, wherein the second end is pulled until the first and second edges of said tissue opening are drawn nearer to one another or in contact with one another.

3. The method of claim 2, wherein the anchor is an adhesive pad that adheres to the luminal tissue wall surface.

4. The method of claim 2, wherein the anchor is an anchor that pierces the luminal tissue wall surface.

5. The method of claim 2, further comprising applying an agent selected from a hemostatic agent, an adhesive agent and a hemostatic adhesive agent to said tissue.

6. A kit for performing the method of claim 2 comprising: (a) said first device and (b) one or more of the following: (i) a tubular sharp-tipped suture delivery device, (ii) a hemostatic agent, adhesive agent or hemostatic-adhesive agent and (iii) one or more tissue closure devices, and (iv) one or more injectable agents to assist with tissue approximation.

7. The method of claim 1, wherein the device selected is said second device and wherein said method comprises (a) pushing a tip of said needle through the tissue wall near a first edge of said tissue opening, (b) pulling the first edge toward a second edge of said opening opposite the first side, and (c) pushing said tip of said needle through the tissue wall near said second edge, such that the needle approximates the first and second edges.

8. The method of claim 7, wherein the second device comprises an elongated member secured to the needle.

9. The method of claim 8, further comprising severing the needle from the elongated member after the needle approximates the first and second edges.

10. The method of claim 7, further comprising applying an agent selected from a hemostatic agent, an adhesive agent and a hemostatic adhesive agent to said tissue.

11. The method of claim 10, wherein said needle is hollow and wherein said agent is applied from an aperture in a side of said needle.

12. A kit for performing the method of claim 7, comprising (a) said second device and (b) one or more of the following: (i) a hemostatic agent, adhesive agent or hemostatic-adhesive agent, (ii) one or more tissue closure devices and (iii) one or more injectable agents to assist with tissue approximation.

13. A device comprising an elongated member and an angled or curved needle secured to the elongated member.

14. The device of claim 13, wherein the needle is angled or curved by at least 180°.

15. The device of claim 13, wherein the needle is detachable from the elongated member.

16. The device of claim 15, wherein the needle is frangible upon application of mechanical or electrical energy.

17. The device of claim 13, (a) wherein the elongated member is a tubular member comprising a first lumen and (b) wherein said needle is hollow and comprises (i) a second lumen in communication with said first lumen and (ii) an aperture in a side of said needle, said aperture connecting the second lumen with an exterior of the needle.

18. A device comprising a suture attached to a tissue anchor.

19. The device of claim 18, wherein the anchor is an adhesive anchor.

20. The device of claim 18, wherein the anchor is tissue-penetrating anchor.
